(19) United States
(12) Patent Application Publication
(10) Pub. No.: US 2010/0068823 A1
(43) Pub. Date: Mar. 18, 2010

(54) Carrier Material, Method for the Production and Use Thereof

(76) Inventor: Thomas Ehben, Weisendorf (DE)

Correspondence Address: HARNESS, DICKEY & PIERCE, P.L.C., P.O.BOX 8910, RESTON, VA, 20195, US

(21) Appl. No.: 11/922,755

(22) PCT Filed: Jun. 21, 2006
(86) PCT No.: PCT/EP2006/063394

§ 371 Date: Dec. 21, 2007

(30) Foreign Application Priority Data
Jun. 27, 2005 (DE) 10 2005 029 808.7

(51) Int. Cl. G01N 33/545; C08F 116/06

(52) U.S. Cl. 436/501; 527/203

(57) ABSTRACT

An embodiment of the present invention relates to a carrier material which is used in a method of diagnosis and which comprises a base material which is provided with a surface which is equipped with at least two different affinity ligands.

### PRIORITY STATEMENT

This application is the national phase under 35 U.S.C. §371 of PCT International Application No. PCT/EP2006/063394 which has an International filing date of Jun. 21, 2006, which designated the United States of America and which claims priority on German Patent Application DE 10 2005 029 808.7 filed Jun 27, 2005, the entire contents of which are hereby incorporated herein by reference.

### FIELD

At least one embodiment of the present invention generally relates to a carrier material for use in a diagnostic method.

### BACKGROUND

Magnetic, polymeric carrier materials, in particular polymer particles, are being increasingly used in biochemistry and medical diagnostics for the separation of cells, proteins and nucleic acids. Compared with conventional separation methods, the use of magnetic carrier materials has the advantage that the loaded carrier materials can simply and rapidly be separated from the other constituents of a sample with the aid of magnetic forces. Magnetic bead-shaped or spherical polymer particles based on polyvinyl alcohol with a narrow particle size distribution within a range of less than 10 μm have proved particularly suitable for such separation methods (WO 97104862).

It is also known that specific biological materials, in particular nucleic acids and proteins, can be isolated from their natural environment only with increased outlay. This is primarily due to the fact that stringent mechanical, chemical and biological cell lysis processes have to be used for isolating the nucleic acids and proteins from the cell nucleus or the cell membrane or organelles. Moreover, the corresponding biological samples usually comprise further, solid and/or dissolved compounds such as other proteins and constituents of the cytoskeleton, which can impair isolation. An additional difficulty is the fact that very often only small concentrations of the nucleic acids or proteins are present in the biological sample to be examined.

In order nevertheless to be able to utilize the advantages of isolating nucleic acids from biological samples using magnetic particles, it has been proposed inter alia to isolate nucleic acids with the aid of magnetic particles having a glass surface which is essentially pore-free (WO 96141811). These particles must have a specific composition, that is to say that their glass surface must have a specific composition, in order to obtain the desired efficacy. Preparation of these particles moreover requires a relatively complicated process to achieve the necessary sintering of the glass surface.

Known diagnostic methods, for example from nucleic acid and protein diagnostics, generally require a multiplicity of manual work steps in order to arrive at an analysis result. This requires inter alia separation of the constituents to be detected from the rest of the sample. Known separation methods are e.g. filtration, centrifugation, chromatography and extraction. These are chemical and physical separation methods which are generally not suitable for specific isolation of DNA or proteins from the sample. By way of example, use is made of resins whose surfaces are functionalized so as to be able to bind DNA or proteins. These target molecules are purified by binding to the solid phase of the resin, followed by a plurality of washing steps and subsequent detachment of the target molecule from the solid phase under suitable buffer conditions.

In this case, the target molecule must be bound tightly, while contaminating constituents of the sample are dissolved in a different, liquid phase. After various washing procedures, the target molecule must then be detached again from the solid phase by changing the liquid phase. The repeated change of medium is firstly very material-intensive, and secondly product yields fluctuate with each additional process step, making quantitative calibration difficult. In particular in integrated analysis methods, for example lab-on-a-chip systems in which the samples are prepared and analyzed as far as possible automatically, checking the individual process steps is often not possible, such that deviations in individual process steps amplify one another and can lead to large deviations in the analysis result.

Individual steps can be simplified or even automated completely by way of the carrier particles described, also called magnetic beads. The magnetic beads are provided with affinity ligands or other surface modifications and are therefore suitable for binding specific biomolecules, for example DNA, from a solution to their surface. A purification method typically involves adding a suspension of magnetic beads to the sample to be separated in a test tube. After an incubation time of a number of minutes in order to enable the affinity ligand to bind to the biological molecule sought, a magnetic field is applied which separates the particles by accumulating them on one wall of the tube. The supernatant is discarded and the particles are washed at least once.

For this purpose, the magnetic field is removed first and the particles are suspended in a fresh buffer solution which contains mainly chaotropic salts which prevent the biomolecules from detaching from the carrier material. The magnetic beads are then deposited on the vessel wall by reapplying the magnetic field. It is thus possible, after a plurality of washing steps, to eluate the molecules in a fresh solution by way of a low salt buffer solution which separates the bound biomolecules from the magnetic beads. The magnetic beads are deposited again on the vessel wall, thereby making available the biomolecules in the supernatant solution. A disadvantage of the method described is the large amount of liquid required in each case, in the range of hundreds of microliters for each individual process step.

For isolating eukaryotic or prokaryotic cells or viruses it is known, by way of example, to couple specific antibodies to a fluorescent marker or magnetic beads. The antibody is generally monoclonal and directed to specific binding sites, for example to a surface receptor molecule of a corresponding antigen of the cell or the virus. As a result of the coupling of the antibodies to the respective binding site, the cells or viruses sought are marked and are sorted for example by way of an FACS (Fluorescence Activated Cell Sorter) or a permanent magnet. In this case, the sorting operation can be carried out on the one hand as a so-called “positive selection”, involving further processing of the marked cells or viruses. On the other hand, a so-called “negative selection” can be carried out, involving removal of the marked cells and further processing of the remaining cells. Both methods enable the cells or viruses to be quantified, such that amounts of reagents required for the further processing can be calculated.

DE 101 11 520 B4 discloses a method for purifying biomolecules with the aid of magnetic particles, in which in particular relatively small amounts of liquid can be purified as far as possible in an automated manner. It describes conveying the suspension with magnetic particles through a pipeline which passes through a strong magnetic field. With suitable settings of diameter, flow rate and magnetic field strength, the magnetic particles are in this case deposited on the wall of the pipeline when flowing through. The supernatant is discarded by emptying the pipeline or is collected in a receptacle. The arrested particles can then be washed by rinsing with washing solutions. During the washing procedure, the magnetic particles can be held in the pipeline or be suspended and deposited again. The biomolecules are separated from the magnetic particles from the suspension by rinsing with a suitable buffer solution. The pipeline here should be configured in such a way that small amounts of liquid of less than 50 μl can also be handled. The method described is suitable in particular for purifying DNA or RNA.

The DNA or RNA available in solution at the end of the method can be introduced into a corresponding analysis system in an automated manner. Automation may be effected by way of a pipetting robot, by way of example. If the DNA is to be detected by way of sequence-specific hybridization, it is moreover proposed to lead the pipeline additionally over a heating device in order to achieve denaturation of the DNA double strand. In order to analyze DNA using the method described, however, it is still necessary to extract the DNA from the sample by way of method steps which have not been described.

Magnetic beads are not only suitable for purifying samples, but can also be used for other purposes. Thus, US 2004/0219066 A1 describes a device which can be used to sort various particles. The particles are bound to different magnetic beads having different magnetic moments. A magnetic field gradient which moves the magnetic beads, owing to their different magnetic moments, into different collecting boxes is generated in a process chamber. The various particles can thus be distinguished by the differently configured magnetic beads.

WO 00/47983 describes an electrochemical biosensor in which magnetic beads are linked via affinity ligands to constituents of a sample. An enzyme is coupled to the bound constituents of the sample and an added substrate is cleaved by the enzyme. The substrate gives rise to a molecule which permits a Redox cycling process. The constituent of the sample can be detected in this way.

It is known, moreover, to use paramagnetic magnetic beads for detecting DNA. In this case, catcher molecules complementary to the DNA to be detected are situated on a magnetorestrictive sensor. If the sample examined contains the DNA to be detected, hybridization takes place between the DNA to be detected and the catcher molecules. The hybridized DNA has been or is marked with a biotin to which streptavidin-coated magnetic beads couple. The biotin marking is generally introduced into the DNA to be detected by way of an upstream PCR utilizing biotin-marked primers. After coupling to the paramagnetic beads, the latter are magnetized by an applied magnetic field and their leakage field is measured by the magnetoresistive sensor. This results indirectly in quantitative detection of the DNA in the sample.

Methods for the production of magnetic polyvinyl alcohol carrier materials, preferably of bead-type particle configuration, are disclosed in DE 41 27 657 and in WO 97104862, the disclosures of which with regard to the methods for the production of carrier materials are hereby incorporated herein by reference. In accordance with the known methods, it is possible to produce magnetic particles with a very narrow particle size distribution and with particle sizes of from 1 to 4 μm, as used in particular for isolating biosubstances in suspension and for diagnostic medicine.

In this case, the polyvinyl alcohol particles are prepared by adding specific emulsifier mixtures to the oil phase of the water-in-oil emulsion. Suitable emulsifiers which are added as additives to the oil phase are propylene oxide-ethylene oxide block copolymers, sorbitan fatty esters, mixed complex esters of pentaerythritol fatty esters with citric acid, polyethylene glycol-castor oil derivatives, block copolymers of castor oil derivatives, polyethylene glycols, modified polyesters, polyoxyethylene sorbitan fatty esters, polyoxyethylene-polyoxypropylene-ethylenediamine block copolymers, polyglyceryl derivatives, polyoxyethylene alcohol derivatives, alkylphenyl-polyethylene glycol derivatives, polyhydroxy fatty acid-polyethylene glycol block copolymers, polyethyleneglycol ether derivatives.

Substances of this type are commercially known inter alia under the trade name: Pluronic®, Synperonic®, Tetronic®, Triton®, Arlacel®, Span®, Tween®, BrijOR, ReneXOR, Hyperme®, Lameform®, Dehymuls® or Eumulgin®.

In order to obtain uniform, bead-shaped polymer particles preferably having particle sizes of 0.5-10 μm, a mixture of at least two, preferably three to four, of the surfactants is added to the oil phase. Preference is given to mixing a lipophilic emulsifier component with at least one emulsifier which has semihydrophilic properties, i.e. which is soluble in both water and oil. Examples of emulsifiers which meet the latter properties are: ethylene oxide-propylene oxide block copolymer derivatives with a predominant ethylene oxide proportion, polyethylene glycol hexadecyl ethers, shorter-chain polyoxyethylene sorbitan fatty esters, polyethyleneglycols or shorter-chain sorbitan fatty esters. The concentration of the emulsifiers in the oil phase is generally 2-6% by volume, preferably 3.5-5.0% by volume. Advantageous with respect to fineness and narrow particle size distribution of polymer droplets are those emulsifier mixtures which comprise at least two lipophilic components and one semihydrophilic emulsifier. The concentration of the semihydrophilic emulsifier is generally between 15 and 30% by volume, based on the total amount of emulsifier. In addition, to fineness of the particles, the particles exhibit a bead-type shape.

Apart from the emulsifiers for the oil phase, special surfactants which are soluble in the aqueous polymer phase also contribute to improving the quality of the emulsion, primarily of polyvinyl alcohol solutions with low molecular weight (Mowiol, Clariant GmbH, Frankfurt am Main, DE). In addition, the magnetic colloids added in solid form are successfully finely dispersed by adding ionic emulsifiers. Examples of such emulsifiers which can also be used as binary mixtures are: serum albumin, gelatin, aliphatic and aromatic sulfonic acid derivatives, polyethylene glycols, poly-N-vinylpyrrolidone or cellulose acetate butyrate. The amounts of emulsifiers used are generally 0.01-2% by weight, based on the polymer phase, with the concentration of the ionic emulsifiers always being between 0.01 and 0.05% by weight. Influences of stirring speeds and concentrations and viscosities of the two phases on particle size are known to the person skilled in the art. In order to realize the preferred particle sizes of 0.5-10 μm, stirring speeds of 1500-2000 revolutions per minute are required, with conventional two-blade propeller stirrers being used.

In principle, those ferro- or superparamagnetic colloids which have an appropriate particle size and generally a magnetic saturation of from 50 to 400 gauss can be used as magnetic particles which are encapsulated into the polyvinyl alcohol matrix during the process. Another requirement to be met by the magnetic particles is dispersibility in the aqueous polymer phase containing the polyvinyl alcohol. During subsequent emulsion in the organic phase, the magnetic colloids are then simultaneously enclosed in the polymer droplets.

Suitable magnetic colloids are preferably magnetites having particle sizes of 10-200 nm. Such substances can be obtained commercially e.g. under the trade name Bayferrox or Ferrofluidics. Since the preparation of such colloids is general prior art, the magnetic particles can also be prepared according to the known methods, as described e.g. by Shinkai et al., Biocatalysis, Vol. 5, 1991, 61, Reimers and Khalafalla, Br. U.S. Pat. No. 1,439,031 or Kondo et al., Appl, Microbiol. Biotechnol., Vol 41, 1994, 99. The concentrations of the colloids in the polymer phase are, in each case based on this phase, generally between 4 and 14% by volume for colloids which are already aqueous colloids due to their preparation, and 0.3-2% by weight for solid substances. Preparation involves admixing the magnetic colloids directly with the polymer phase. In order to ensure a finely dispersed, uniform distribution of the particles, brief mixing of the aqueous dispersion by way of a high revolution dispersing tool (Ultra-Turrax) with subsequent ultrasound treatment is beneficial. The polymer phase required for preparing the magnetic particles generally comprises a 2.5-10% by weight polyvinyl alcohol solution.

The magnetic polyvinyl alcohol carrier material can then be obtained from the suspension according to the methods known per se to the person skilled in the art, for example by filtration and washing.

A known process for functionalization comprises equipping the carrier material with affinity ligands on the surface. This generally requires attaching chemically reactive groups on the surface, to which the affinity ligands are then bound. These groups may be embodied for example as tosyl, hydroxyl, aldehyde or carboxyl, amino, thiol or epoxy groups. They can generally be provided by treating uncoated monodisperse superparamagnetic particles in order to provide them with a surface layer of a polymer carrying such a functional group, for example a cellulose derivative or a polyurethane together with a polyglycol for providing hydroxyl groups, a polymer or copolymer of acrylic acid or methacrylic acid for providing carboxyl groups or an amino-alkylated polymer for providing amino groups. U.S. Pat. No. 4,654,267 discloses a plurality of surface coatings.

DE 100 13 995 A1 discloses magnetic carrier materials based on polyvinyl alcohol, the surface of which is at least partly silanized and, if appropriate, equipped with biomolecule-coupling affinity ligands. The carrier materials described may be configured as filter, membrane or particle. The magnetic carrier material is preferably in the form of bead-shaped or spherical particles, the particles having a particle size preferably of from 0.2 to 50 μm, particularly preferably from 0.5 to 5 μm. Aside from the preferably bead-shaped and spherical configuration of the particles, their particle size distribution ought to be within as narrow a range as possible. The carrier materials are preferably prepared in particle form by reacting the polyvinyl alcohol carrier material with an organic silane compound. The silanized particles are then reacted with affinity ligands.

Affinity ligands which may be coupled are in principle all ligands used in affinity chromatography. Examples thereof are: protein A, protein G, protein L, streptavidin, biotin, heparin, antibodies, serum albumin, gelatin, lysine, concanavaline A, oligosaccharides, oligonucleotides, polynucleotides, protein-binding metal ions, lectins, aptamers or enzymes. The special fractionations which can be carried out using such affinity matrices are general prior art.

### SUMMARY

In at least one embodiment, the present invention provides improved carrier materials which permit largely automatic diagnostic methods.

In at least one embodiment, he carrier material includes a base material having a surface which is equipped with at least two different affinity ligands. This should be understood to mean different types of affinity ligands, rather than a plurality of specimens of one type of affinity ligands. Equipping the carrier material with at least two different affinity ligands increases the spectrum of use in comparison with the known embodiments. In particular, affinity ligands of the types mentioned above can be used in this case. In general, a multiplicity of specimens of each type of affinity ligands will be bound on the surface.

The carrier material according to at least one embodiment of the invention can be used within a diagnostic method for different tasks, in particular tasks that are to be carried out successively, such that manual work steps can be automated as far as possible. Diagnostic methods can be simplified as a result. Known carrier materials having in each case only one type of affinity ligands are suitable for example only for binding one cell type. In a complex analysis method in which a plurality of different cell types are to be detected, or in which DNA obtained from a cell is processed further, different carrier materials are consequently required. There is the problem here that carrier materials that have already been used and are no longer required are taken along into subsequent process steps and interfere with the latter. This is problematic particularly in the case of magnetic or magnetizable carrier materials, since it is particularly easy for the latter to be taken along and have an interfering effect.

The carrier material according to at least one embodiment of the invention affords the advantage here that the carrier materials can be used in different process steps for example while an analysis is being carried out. By virtue of the multiple use of the carrier material according to the invention, no excess carrier material that has already been used in one process step is taken along into subsequent process steps. The carrier material according to the invention makes it possible to avoid process steps in which excess carrier material is removed from the process.

In one embodiment, the affinity ligands are chosen in such a way that a first one of the affinity ligands has binding properties for a biological structure and a second one of the affinity ligands has binding properties for a biological molecule extracted from the biological structure.

The term “biological structure” should be understood hereinafter to mean in particular bacteria, cells and viruses. However, it can also mean other biological structures of a sample, such as, for example, proteins, peptides, spores, chromosomes, protozoa or other constituents of the sample. The term “biological molecule” should be understood hereinafter to mean primarily DNA, RNA, proteins, carbohydrates and lipids. In general, it should be understood to mean any types of molecules which are to be detected for example within an analysis of the sample. These also include organic and inorganic toxins, for example.

By correspondingly equipping the carrier material, it is possible to manipulate both structures and molecules with a carrier material within a diagnostic method. It is often the case that biological structures, for example, are initially present in a sample and a molecule is then extracted from them. The molecule is bound by the carrier material and can be correspondingly manipulated or purified.

In one advantageous configuration of at least one embodiment of the invention, the surface is equipped with further affinity ligands having binding properties for further biological structures. This is important particularly for diagnostic methods in which a molecule occurs in different structures. Thus, using just one carrier material, the corresponding different structures can be extracted from the sample and purified. After the molecules have been extracted, they can be processed further.

In one advantageous configuration of at least one embodiment of the invention, the surface is equipped with further affinity ligands having binding properties for further biological molecules. This is important in methods in which different molecules occur in different specimens of a structure. Accordingly, after the different molecules have been extracted from the specimens of the structure, the molecules can be processed further with the aid of the carrier material.

In a particularly advantageous configuration of at least one embodiment of the invention, the first one of the affinity ligands has binding properties for a specific protein and the second one of the affinity ligands has binding properties for a specific nucleic acid sequence. This is of interest in particular in a method for detecting the nucleic acid sequence. The nucleic acid sequence may characterize a specific bacterium, for example. The bacterium can be detected by detection of the nucleic acid sequence in an analysis method. The nucleic acid sequence is typically present within the bacterium.

In the analysis method of at least one embodiment, by way of example, firstly the bacterium is bound to the first affinity ligand via a cell receptor. After cell disruption involving the liberation of the nucleic acid sequence, the latter binds to the second affinity ligand and can thus be manipulated by way of the carrier material. In the case of a magnetic embodiment of the carrier material, it is also possible, as has already been described in the introduction, to detect the nucleic acid sequence with the aid of the carrier material. In this case, it is particularly important that no excess magnetic carrier materials from previous process steps are present which might have an interfering influence on the magnetic detection.

In one advantageous embodiment of the invention, the first affinity ligand is embodied as an antibody or a part of an antibody and the second affinity ligand is embodied as an oligonucleotide. Oligonucleotides should be understood hereinafter to mean single-stranded nucleic acid molecules. This embodiment of the affinity ligands can be prepared particularly simply and specifically and be applied to the surface of the base material. At the same time, it affords highly specific binding properties for biological structures and DNA, for example, such that a high degree of specification is achieved. In this case, oligonucleotides having different base sequences should be interpreted as different affinity ligands since they have binding properties for different DNA sequences.

In one advantageous embodiment of the invention, the base material contains paramagnetic particles. The carrier material can be formed as magnetic beads, by way of example. The manipulability of the magnetic beads affords the advantage that it becomes possible for example to move the target structures bound to the affinity ligands, that is to say DNA or cells, for example, by way of magnetic forces. As has already been explained, it is also possible to detect DNA, for example, by way of the magnetic beads.

The method according to at least one embodiment comprises the following method steps:
applying chemically reactive groups to a surface of a base material,
introducing the base material into a first coating solution, in which first affinity ligands are present,
binding the first affinity ligands to a first portion of the chemically reactive groups,
introducing the base material into a second coating solution, in which second affinity ligands are present, and
binding the second affinity ligands to a second portion of the chemically reactive groups.

By progressively introducing the base material into the different coating solutions, it is possible to apply any desired affinity ligands to the surface of the base material. It is important here that in all cases only a portion of the reactive groups is covered with affinity ligands, in order that further types of ligands can be applied.

In one advantageous embodiment of the method according to at least one embodiment of the invention, the base material is introduced into further coating solutions, in which respective affinity ligands are present which are bound to further portions of the chemically reactive groups. It is thus possible to produce multifunctional carrier materials for a diagnostic method in a simple manner.

In one advantageous embodiment of the method according to the invention, the base material is introduced into a further coating solution, in which proteins are present which bind to the as yet unoccupied chemically reactive groups. This prevents uncovered reactive groups from reacting with constituents of the sample during a diagnostic process.

The method according to at least one embodiment comprises the following method steps:
applying chemically reactive groups to a surface of a base material,
introducing the base material into a coating solution in which at least two affinity ligands are present,
binding the affinity ligands to a respective portion of the chemically reactive groups.

This alternative production method likewise permits a simple possibility for the production of the carrier materials according to the invention. In contrast to the method according to claim 15, a mixture is employed here, such that the carrier material is coated with different affinity ligands in one method step.

The carrier material is preferably used for a nucleic acid analysis, nucleic acid preparation and/or a nucleic acid detection. The use of the carrier material according to at least one embodiment of the invention affords advantages particularly in the magnetic detection of nucleic acids.

The same applies to the use of the carrier material for a protein analysis.

### BRIEF DESCRIPTION OF THE DRAWINGS

Further advantages of the invention are explained on the basis of the example embodiment described below in connection with the accompanying drawings, in which:

FIG. 1 shows a schematic illustration of an already known carrier material,

FIG. 2 shows a schematic illustration of a further already known carrier material,

FIG. 3 shows a schematic illustration of an example embodiment of the invention,

FIG. 4 shows a schematic illustration of an alternative example embodiment of the invention, and

FIG. 5 shows a schematic flow diagram of a method for the production of the carrier materials.

### DETAILED DESCRIPTION OF THE EXAMPLE EMBODIMENTS

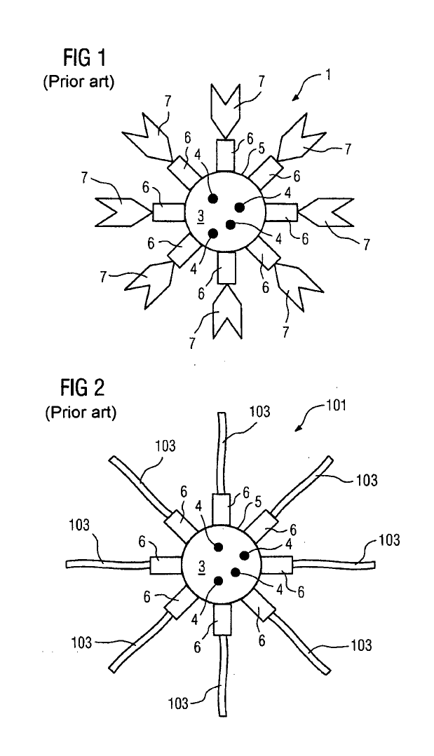

FIG. 1 schematically illustrates a carrier material 1. It is known per se in the form shown and is preferably used in diagnostic processes. It comprises a magnetic bead 3 produced for example according to the method described above. The magnetic bead 3 contains a plurality of superparamagnetic particles 4. A surface 5 of the magnetic bead 3 is implemented with reactive groups 6. Antibodies 7 are bound to the reactive groups 6. The antibodies 7 have binding properties for a biological structure, that is to say for example a eukaryotic cell, a bacterium or a virus, which is present in a sample to be analyzed. By virtue of the antibodies 7, the carrier material 1 is able to bind the biological structure to itself, whereby the structure can be manipulated by way of a magnetic field. In this case, the superparamagnetic particles 4 only become magnetic if a magnetic field is present. Likewise, the magnetic properties are lost again as soon as the magnetic field is switched off. This prevents a clumping of the individual magnetic beads 3, for example, which are generally present in a large number. As has already been described, the biological structures sought can be separated from the sample by the use of the magnetic field.

FIG. 2 shows a further carrier material 101, which is likewise known. Carrier materials 101 of this type are preferably used in nucleic acid diagnostics. The carrier material 101 is constructed in a manner similar to the carrier material 1 shown in FIG. 1 and includes a magnetic bead 3. Reactive groups 6 are arranged on the surface 5 of the magnetic bead 3 and oligonucleotides 103 are bound to the groups as affinity ligands. Each of the oligonucleotides 103 is directed toward a DNA to be detected, such that when the DNA is present in a sample, hybridization can take place between the DNA and the oligonucleotides. The DNA to be detected originates for example from a virus or a cell and is generally replicated for detection by way of a PCR. By linking the DNA to the carrier material 101, separation from the rest of the sample is possible. Likewise, direct detection of the DNA is possible by way of the magnetic property of the magnetic bead 3, as already described.

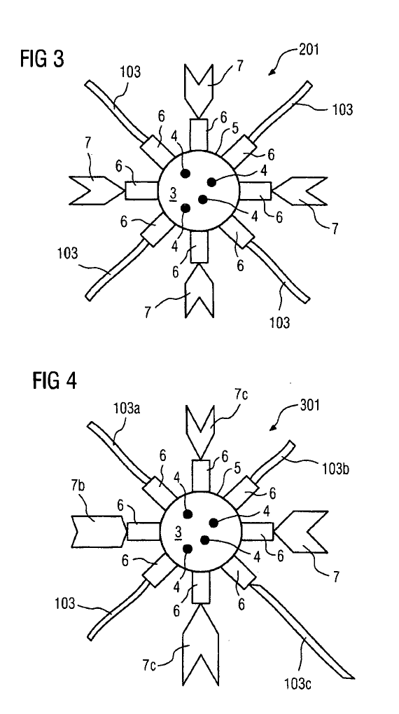

FIG. 3 schematically illustrates an example embodiment of the invention. Analogously to the embodiments already known, a carrier material 201 comprises a magnetic bead 3, on the surface 5 of which are arranged reactive groups 6 with affinity ligands. In the present example embodiment, both antibodies 7 and oligonucleotides 103 are provided on the magnetic bead 3. The carrier material 201 functionalized in this way can be used for different purposes in a correspondingly configured analysis or diagnostic process. The antibodies 7 are directed for example toward a cell receptor (e.g. CD4) of one cell type, that is to say are preferably monoclonal antibodies. The oligonucleotides 103 are directed toward a gene sequence of the cell, for example toward an activated gene in T helper cells.

Thus, by utilizing the antibodies 7 it is possible for example to separate the cells sought from the sample. After cell disruption, the DNA is present in free form and can bind to the oligonucleotides 103 after denaturation and possible comminution. The bound DNA can be separated from the sample and detected according to known methods, for example by a GMR or TMR sensor. Consequently, the carrier materials according to an embodiment of the invention and its embodiments make it possible to simplify such diagnostic and analysis methods.

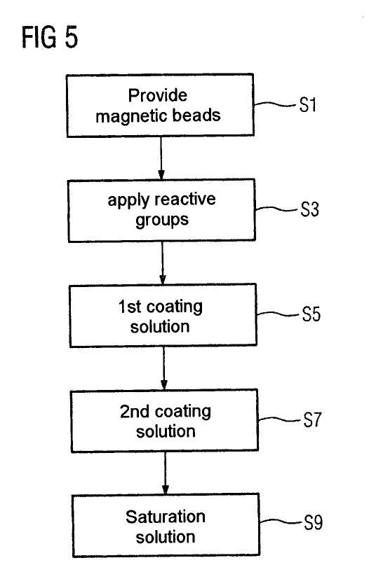

FIG. 4 shows an alternative embodiment of the invention. Analogously to the embodiments described above, the carrier material 301 comprises a magnetic bead 3, on the surface of which are arranged reactive groups 6 and affinity ligands. In this embodiment, different antibodies 7, 7a, 7b and 7c are bound to one portion of the groups 6. The antibodies 7, 7a, 7b and 7c are directed toward different types of biological structures. These may be for example different cells or viruses. Different oligonucleotides 103, 103a, 103b and 103c are bound to another portion of the groups 6. The oligonucleotides 103, 103a, 103b and 103c are directed toward gene sequences of the structures which can be selected by the antibodies 7, 7a, 7b and 7c. As a result, a plurality of cell types and the DNA thereof can be isolated and detected with just one type of carrier material.

Further embodiments (not illustrated here) are also possible in addition to those already described. Thus, a gene sequence of interest may occur in a plurality of cells, for example 16S rRNA from different bacteria. A carrier material having oligonucleotides for the gene sequence and antibodies for the appropriate cell types is suitable for enabling the isolation and the detection in an analysis method in a simple manner. It likewise happens that different gene sequences or SNPs occur in one cell type and are to be detected. A corresponding carrier material comprises corresponding antibodies for binding the cell and different oligonucleotides for the gene sequences or SNPs.

As a result of using the example embodiments described in analysis or diagnostic methods, the latter can be carried out more simply than known methods. In particular, the changes of analytes that are often necessary when using a plurality of types of carrier materials can be avoided. Elutions and rinsing operations are thus obviated. In methods with a plurality of types of carrier materials it happens that magnetic beads that are no longer required per se influence the further method and in particular the detection of the DNA. Particularly in the case of detection processes in which the magnetic beads serve as markers for attached molecules, the signal of the magnetic beads still present from previous method steps can interfere with the detection process and corrupt the actual signal. The problem can be prevented by using only one type of carrier material according to the invention or one of the example embodiments.

FIG. 5 illustrates a schematic flow diagram of a method for the production of carrier materials. The production of the magnetic beads as base material is general prior art and has already been described in the introductory stages. Accordingly, the magnetic beads are provided in a first method step S1. In a second method step S3, chemically reactive groups are applied to the magnetic beads, which is likewise effected according to methods known per se. Examples of appropriate reactive groups include tosyl, carboxyl, amino or epoxy groups. In further method steps S5 and S7, the magnetic beads are provided with affinity ligands in different coating solutions. In this case, the chemically reactive groups bind covalently with the affinity ligands present in the coating solution. After method step S7, it is possible to pass through even further coating solutions until the surface of the magnetic beads has been functionalized in accordance with the specifications. In this case, the concentration of the affinity ligands in the coating solutions should be chosen such that after the magnetic beads have been introduced into the coating solution, rather than the complete surface of the magnetic beads only a corresponding fraction is covered with the respective affinity ligands. Thus, there is still space remaining on the surface for the further coating steps.

As an alternative, it is possible to completely functionalize the magnetic beads in a single coating solution. In the coating solution, the desired affinity ligands are present in a mixture, such that all the affinity ligands simultaneously bind to the surface of the magnetic beads. In this case, it is possible to use chemically reactive groups which bind with one specific, but not another affinity ligand. It is ensured in this way that the ratio of the different affinity ligands on the surface of the magnetic beads can be controlled.

In both possible methods it is possible that not all the reactive groups are covered with affinity ligands after the conclusion of the coating. This could lead to problems in an analysis process as soon as the magnetic beads come into contact with a sample. The groups would react with constituents of the samples, which could impede the course of the process or influence the result. For this reason, the magnetic beads are brought into a saturation solution in a further method step S9. The saturation solution contains proteins in high concentration which bind to the still free reactive groups and thus cover them. This prevents the groups from reacting with sample constituents.

The magnetic beads described can be used for example in methods for the detection of specific nucleic acid sequences. So-called lab-on-a-chip systems are increasingly gaining in importance here. Systems of this type often include a single-use cartridge in which the sample is processed and analyzed in different process chambers connected by microchannels. A control unit into which the cartridge is inserted controls the analysis process in the cartridge.

By way of the magnetic beads described, novel lab-on-a-chip systems can be defined and correspondingly novel analysis methods can be implemented. A description is given below by way of example of an advantageous, as far as possible automated analysis method in which the multifunctional magnetic beads provided can advantageously be used. Specific cells in a sample are intended to be detected by way of the method, in order thus to produce a diagnosis.

In a first method step, the patient's sample is introduced through a filling opening in the cartridge. The cartridge is thereupon inserted into the control unit, whereby the analysis process starts automatically. In a second method step, in a preparation chamber, the magnetic beads stored therein bind to the sample cells to be examined. In this case, the magnetic beads in the solution are moved to and fro by suitable manipulation of a magnetic field in order to accelerate the operation. In a third method step, the process chambers of the cartridge are filled with water and the reagents stored in dry form therein are dissolved. In a fourth method step, the magnetic beads are moved into a structure disruption chamber through the magnetic field. In a fifth method step, by way of a lysis buffer which is stored in the structure disruption chamber and is present in solution as a result of the flooding with water, the cells bound to the magnetic beads are dissolved and the DNA contained in them is liberated.

The DNA is denatured by brief heating of the lysis buffer. As an alternative, sodium hydroxide solution can also be added to the lysis buffer, which likewise results in a denaturation. The liberated DNA molecules bind to the magnetic beads. At the same time, the antibodies are detached from the surface of the magnetic beads by a protease enzyme in the lysis buffer, such that residues of the cell structures are no longer attached to the magnetic beads either. Consequently the magnetic beads are only linked with the DNA molecules of the cells to be analyzed.

In a sixth method step, the magnetic beads with DNA molecules are moved through a microchannel into a washing chamber of the device. In a seventh method step, cell residues that are possibly present and any other impurities are washed out. In an eighth method step, the magnetic beads are moved into an amplification chamber. In a ninth method step, a polymerase chain reaction is carried out in the amplification chamber and the DNA of the cells to be examined is thereby replicated. In order to carry out the chain reaction, a plurality of thermal cycles between two temperatures are carried out in the amplification chamber by way of a Peltier element of the control unit.

In a tenth method step, the magnetic beads and the DNA fragments bound thereto are moved through a microchannel into a detection chamber of the cartridge, in which hybridization of the DNA fragments to oligonucleotides arranged in the detection chamber takes place in an eleventh method step. In this case, on account of the specific binding properties, only those DNA fragments which are intended to be analyzed are attached to the oligonucleotides. Consequently, these are specifically tailored to the analysis of a specific cell type. Finally, the hybridization is detected for example by magnetic detection of the magnetic beads. During this detection method, in particular, magnetic beads possibly present from previous process steps and no longer required would lead to problems due to their magnetic leakage field. The multifunctional magnetic beads provided afford major advantages here.

In an alternative method, even further purification steps may be provided, for example in a further washing chamber arranged between the preparation chamber and the structure disruption chamber. It is additionally possible to arrange a plurality of washing chambers in succession in order to be able to perform a plurality of washing steps one after another.

The method described above relates only to one type of DNA to be detected, for example from a specific virus. However, the method steps can also be parallelized in such a way that different types of DNA can be detected. Correspondingly prepared magnetic beads and corresponding detection possibilities should then be provided. It is likewise necessary to orientate the PCR to a plurality of types of DNA.

It is likewise possible to include RNA in the analysis process. Prior to amplification, the RNA is converted into so-called cDNA by reverse transcription and can then be replicated by way of PCR and detected by the detection unit.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A carrier material for use in a diagnostic method, comprising:
a base material including a surface with at least two different affinity ligands, a first one of the affinity ligands having binding properties for a biological structure and a second one of the affinity ligands having binding properties for a biological molecule extracted from the biological structure.

2. The carrier material as claimed in claim 1, wherein the surface is equipped with further affinity ligands having binding properties for further biological structures.

3. The carrier material as claimed in claim 1, wherein the surface is equipped with further affinity ligands having binding properties for further biological molecules.

4. The carrier material as claimed in claim 1, wherein the first one of the affinity ligands has binding properties for a specific protein and the second one of the affinity ligands has binding properties for a specific nucleic acid sequence.

5. The carrier material as claimed in claim 1, wherein the first affinity ligand is embodied as an antibody or a part of an antibody and the second affinity ligand is embodied as an oligonucleotide.

6. The carrier material as claimed in claim 1, wherein the base material is based on a polymer.

7. The carrier material as claimed in claim 6, wherein the polymer is polyvinyl alcohol.

8. The carrier material as claimed in claim 1, wherein the base material is present in the form of spherical particles.

9. The carrier material as claimed in claim 8, wherein the particles have a size of 10 nm to 50 μm.

10. The carrier material as claimed in claim 1, wherein the base material contains paramagnetic particles.

11. The carrier material as claimed in claim 1, wherein the surface of the base material is coated.

12. The carrier material as claimed in claim 1, wherein chemically reactive groups to which the affinity ligands are bound are arranged on the surface.

13. The carrier material as claimed in claim 12, wherein the chemically reactive groups are selected from the group consisting of tosyl, carboxyl, amino, thiol and epoxy groups.

14. The carrier material as claimed in claim 2, wherein the surface is equipped with further affinity ligands having binding properties for further biological molecules.

15. The carrier material as claimed in claim 2, wherein the surface is equipped with further affinity ligands having binding properties for further biological molecules.

16. The carrier material as claimed in claim 2, wherein the first one of the affinity ligands has binding properties for a specific protein and the second one of the affinity ligands has binding properties for a specific nucleic acid sequence.

17. The carrier material as claimed in claim 2, wherein the first affinity ligand is embodied as an antibody or a part of an antibody and the second affinity ligand is embodied as an oligonucleotide.

18. The carrier material as claimed in claim 2, wherein the base material is based on a polymer.

19. A method for the production of a carrier material, comprising:
applying chemically reactive groups to a surface of a base material;
introducing the base material into a first coating solution, in which first affinity ligands are present;
binding the first affinity ligands to a first portion of the chemically reactive groups;
introducing the base material into a second coating solution, in which second affinity ligands are present; and
binding the second affinity ligands to a second portion of the chemically reactive groups.

20. The method as claimed in claim 19, wherein the base material is introduced into further coating solutions, in which respective affinity ligands are present, which are bound to further portions of the chemically reactive groups.

21. The method as claimed in claim 19, wherein the concentration of the affinity ligands in each of the coating solutions is such that the respective affinity ligands are bound only to the corresponding portion of the chemically reactive groups.

22. The method as claimed in claim 19, wherein the base material is introduced into a further coating solution, in which proteins are present which bind to the as yet unoccupied chemically reactive groups.
